(19) United States
(12) Patent Application Publication
(10) Pub. No.: US 2020/0023042 A1
(43) Pub. Date: Jan. 23, 2020

(54) COMPOSITION FOR WOUND HEALING

(71) Applicant: GEORGE D. PETITO, BETHLEHEM, PA (US)

(72) Inventor: GEORGE D. PETITO, BETHLEHEM, PA (US)

(21) Appl. No.: 16/504,643

(22) Filed: Jul. 8, 2019

Related U.S. Application Data
(60) Provisional application No. 62/699,400, filed on Jul. 17, 2018.

(51) Int. Cl. A61K 38/39; A61K 35/20; A61K 47/12; A61K 31/726; A61K 31/727; A61K 31/728; A61K 45/06; A61K 31/198; A61K 33/30; A61K 31/734; A61K 31/555; A61K 38/36; A61K 33/38; A61K 31/717; A61K 35/644; A61K 47/10; A61K 31/65; A61K 31/7048; A61K 31/545; A61K 33/18; A61K 38/44; A61K 36/71; A61K 36/605

(52) U.S. Cl. CPC A61K 38/39; A61K 36/605; A61K 47/12; A61K 31/726; A61K 31/727; A61K 31/728; A61K 45/06; A61K 31/198; A61K 33/30; A61K 31/734; A61K 31/555; A61K 38/363; A61K 33/38; A61K 31/717; A61K 35/644; A61K 47/10; A61K 31/65; A61K 31/7048; A61K 31/545; A61K 33/18; A61K 38/446; A61K 38/44; A61K 36/71; A61K 35/20

(57) ABSTRACT

The composition for wound healing facilitates healing of damaged tissues, promotes tissue and cell growth, protects cells and tissues, and reduces scar tissue. The composition includes hydrolyzed collagen, whey, and magnesium stearate. The composition for wound healing demonstrates improved flow, increasing the ease of manufacturing and application to a wound site. The hydrolyzed collagen may include bovine sourced hydrolyzed collagen and marine sourced hydrolyzed collagen. The composition may also include an additive, such as a therapeutic agent, an antimicrobial, a vitamin, or a preservative.

### CROSS-REFERENCE TO RELATED APPLICATION

This application claims the benefit of U.S. Provisional Patent Application Ser. No. 62/699,400, filed Jul. 17, 2018.

### BACKGROUND

### 1. Field

The composition for wound healing relates to wound healing, and particularly to a method and composition for wound healing in animals or humans that provides for administering a composition comprising hydrolyzed collagen and magnesium stearate.

### 2. Description of the Related Art

Just as nature has provided the skin as a barrier for protection it has also provided mechanisms for skin repair. Depending upon the nature of the injury, this repair process may take hours, days, months, or even years. Many factors determine the length of time it takes for injured skin to heal. Pathogenic contaminants may enter the body through the wound until the skin's integrity is restored. For this reason, it is desirable to heal open wounds as quickly as possible.

Open wounds in the skin are a potential gateway for infectious or contaminating material to enter the body. The skin is a protective barrier to external contaminants. When the skin is damaged with an open breach, these contaminants are free to enter the body. Once inside the body, these contaminants may have effects of varying degrees, but almost always become more difficult to treat, and consequently slow the process of healing the original wound.

In order to fight infection, wound management traditionally involves an initial cleansing of the affected area to remove any contaminants, such as dirt, clothing particles, or other debris. Damaged tissue and foreign materials are removed when necessary, and antiseptic agents are applied to sterilize the injured area. Sterile dressings are often applied, and are periodically changed to keep the injured area as clean and sterile as possible. Complex biological mechanisms occur during the healing process, such as chemical signals attracting fibroblast cells to the wound site, which ultimately generate connective structures, mainly of collagen. Endothelial cells generate new blood capillaries that nurture the new growth. Cell growth continues until the open wound is filled by forming permanent new tissue.

Recently, compositions containing collagen and hydrolyzed collagen have demonstrated improved wound healing capabilities. However, the manufacture and application of these products can be challenging. Particularly where specific mixtures of different collagen components are desired, the viscosity of the composition during manufacture may lead to mix variations and flow problems through critical equipment, causing delays, loss of product, and increased cost of production.

Thus, a composition for wound healing solving these problems is desired.

### SUMMARY

A method and composition for wound healing facilitates healing of damaged tissues, promoting tissue and cell growth, protecting cells and tissues, and reducing scar tissue. The composition includes proteinaceous amino acids, including hydrolyzed collagen, whey, and magnesium stearate. In some applications, the composition may include additives, such as therapeutic agents, antimicrobials, vitamins, or preservatives. In an embodiment, the hydrolyzed collagen may include one or more of bovine derived hydrolyzed collagen and marine derived hydrolyzed collagen.

These and other features of the composition for wound healing will become readily apparent upon further review of the following specification.

### DETAILED DESCRIPTION OF THE PREFERRED EMBODIMENTS

The method and composition for wound healing facilitates healing of damaged tissues, promoting tissue and cell growth, protecting cells and tissues, and reducing scar tissue. The composition includes proteinaceous amino acids, including hydrolyzed collagen, whey, and magnesium stearate. The proteinaceous amino acids may facilitate tissue and cell growth as well as wound healing. The magnesium stearate acts as a lubricant, improving the flow of the composition. This improved flow renders the composition both easier to manufacture (easier to manipulate into storage vessels such as tubes for resale) and easier to apply to a sensitive wound site.

In some applications, the composition may include additives, such as therapeutic agents, antimicrobials, vitamins, or preservatives. In an embodiment, the hydrolyzed collagen may include one or more of bovine derived hydrolyzed collagen and marine derived hydrolyzed collagen.

As used herein to define Daltons, the term “about” shall mean within 5,000 Daltons. As used herein to define a weight by volume percentage, the term “about” shall mean within 1% of the specified concentration.

Hydrolyzed collagen is a collagen hydrolysate polypeptide having a molecular weight lower than native collagen. Hydrolyzed collagen may be obtained by hydrolysis of native collagen. This may be accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from a collagen source.

The hydrolyzed collagen can be derived from any suitable collagen source. The collagen source can be, for example, a bovine (skin and tendon preferred), a porcine, a reptile, a marine, an avian, an equine, and/or a synthetic source. For example, the collagen can be derived from a combination of two or more collagen sources, e.g., a bovine source and a marine source. The marine source can include any fish. Preferably, the marine source includes salmon, tilapia, or a combination of salmon and tilapia.

The types of amino acid constituents and their sequences determine the beneficial healing qualities of hydrolyzed collagen. Hydroxylysine and hydroxyproline are amino acids found only in collagen and in no other medical protein hydrolysates. Hydroxylysine is typically found in concentrations of from about 0.7 to about 1.2 wt. % in hydrolyzed collagen.

Bovine and porcine hydrolyzed collagens have high glycine, proline, hydroxyproline, and glutamic acid content. They also display hydrophilic properties. Bovine hydrolyzed collagen, for example, demonstrates strong hydrophilic properties and when used to treat wound sites, demonstrates increased perfusion and epithelialization and decreased inflammatory reaction. In contrast, marine derived, i.e., marine sourced, hydrolyzed collagen has a different amino acid profile, with higher levels of aspartic acid, cysteine, glutamine, citruline, and asparagine.

Hydrolyzed whey protein offers another alternative amino acid profile, rich in glutamic acid, isoleucine, leucine, threonine, tyrosine, and valine.

Magnesium stearate functions primarily as a lubricant, and is understood to reduce inter-particle friction. The use of magnesium stearate improves the consistency of the final product and enhances flow through the manufacturing process.

Preferably, the composition includes hydrolyzed Type 1 collagen. While hydrolyzed collagen of any molecular weight may be used, the hydrolyzed collagen can be high molecular weight hydrolyzed collagen, hereinafter “HMW hydrolyzed collagen,” having a molecular weight less than native collagen. For example, the HMW hydrolyzed collagen may have a molecular weight of from about 10,000 to about 300,000 Daltons, particularly from about 10,000 to about 95,000 Daltons. That is to say, the HMW collagen may have a molecular weight of about 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, or 300,000 Daltons.

In an embodiment, the composition can include mixtures of collagen from different collagen sources. For example, the composition can include bovine sourced collagen, marine sourced collagen, and whey protein. According to another embodiment, the proteinaceous amino acids in the composition can include bovine sourced hydrolyzed collagen, marine sourced hydrolyzed collagen, and hydrolyzed whey protein. The composition can further include elastin.

The composition may include about 30% by weight to about 90% by weight hydrolyzed collagen. For example, the composition may include about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, or about 90% by weight hydrolyzed collagen.

One or more additional therapeutic agents may be included in the composition to further speed the healing process, decrease scarring and increase tissue strength. Examples of suitable therapeutic agents that may be combined with the hydrolyzed collagen are glycosaminoglycans (GAGs), particularly GAGs useful for cellular repair. Antimicrobials may also be included in the composition to further enhance its bacteriostatic quality, as can antibiotics (such as tetracycline, streptomycin, and cephalosporin) and antibacterials (such as iodine, parachlorometaxylenol, ortho-phthalaldehyde, iodophors, quaternary ammonium compounds, and chlorhexidine gluconate or acetate). The composition may further include lipoic acid, one or more vitamins (e.g., vitamin A, vitamin B12, vitamin C, vitamin E), omega compounds or omega-3 fatty acid compounds (e.g., ALA, EPA, DHA), antioxidants (e.g., superoxide dismutase, glutathione peroxidase, glutathione reductase), and/or phytochemicals (e.g., zeaxanthin, lutein). Also, it has been established that hydrolyzed collagen used as a carrier in powder form, paste or a lyophilized foam has hemostatic qualities when combined with thrombin to improve healing of wounds.

Glycosaminoglycans (GAGs) are polysaccharides found in vertebrate and invertebrate animals. Several GAGs have been found in tissues and fluids of vertebrate animals. The known GAGs are chondroitin sulfate, keratin sulfate, dermatic sulfate, hyaluronic acid, heparin, and heparin sulfate. GAGs and collagen are the major structural elements of all animal tissue. Their synthesis is essential for proper repair, treatment, protection, and maintenance of all tissues.

A particularly preferred glycosaminoglycan is chondroitin sulfate, a polysulfated GAG. Chondroitin sulfate is a linear polymer occurring in several isomers, named for the location of the sulfate group. Chondroitin-4 sulfate is found in nasal and tracheal cartilages of bovines and porcines. It is also found in the bones, flesh, blood, skin, umbilical cord, and urine of these animals. Chondroitin-6 sulfate has been isolated from the skin, umbilical cord, and cardiac valves of the aforementioned animals. Chondroitin-6 sulfate has the same composition, but slightly different physical properties from the chondroitin-4 sulfate. These are the most common isomers used in the present composition. The polymers are also known as polysulfated glycosaminoglycans (PSGAGs), chondroitin polysulfate sodium, chondrin, sodium chondroitin polysulfate, and sodium chondroitin sulfate. For consistency, the term “chondroitin sulfate” will be recited for all chondroitin sulfate isomers throughout this specification. Chondroitin sulfate is involved in the binding of collagen, and is also directly involved in the retention of moisture in the tissue. These are both valuable chemical properties that aid the healing process.

Hydrolyzed collagen in combination with GAGs, specifically a PSGAG (such as chondroitin sulfate), can be useful for the prevention and treatment of wound diseases. The hydrolyzed collagen combines with a PSGAG to bond or adhere selectively to tissue, resulting in interference with and/or displacement of bacterial or other infectious agents. In addition, the combination product may exhibit anti-enzyme activity or the ability to inhibit enzyme activity.

Hyaluronic acid and other proteoglycans (PGs) can be included in the composition. The hydrolyzed collagen accelerates the healing process by allowing an injured tissue to repair itself by producing and remodeling more collagen and other proteoglycans (PGs). The building blocks for collagen production are the amino acids found in hydrolyzed collagen. Hyaluronic acid and other proteoglycans (PGs) provide the framework for collagen production to follow. The PGs hold water to provide an excellent environment for healing of the tissue to begin. When in the wound site, any unused collagen that was produced is simply degraded to the amino acid. The rate-limiting step in the production of collagen is the conversion of glucose to glucosamine for the production of hyaluronic acid and other glycosaminoglycans (GAGs).

The composition can include one or more therapeutic agents, such as an antibiotic, and/or one or more additives, such as glutamine, glycosaminoglycans, zinc, alginates, copper chlorophillan, fibronectin, silver, oxidized regenerated cellulose, cellulose, and/or honey.

The composition can include one or more cross-linking agents, such as, humectant, propylene glycol, sorbitol, and glycerine.

Further examples of this wound healing composition optimized for different applications include: about 30% to about 90% bovine sourced hydrolyzed collagen, about 20% to about 50% marine sourced hydrolyzed collagen; about 0.5% to about 60% whey, and magnesium stearate. In a preferred embodiment, the marine sourced collagen may be sourced from salmon and the whey may be hydrolyzed whey. For example, the composition may include about 30%, about 35%, about 40%, about 45%, about 50%, about 55%, about 60%, about 65%, about 70%, about 75%, about 80%, about 85%, or about 90% by weight bovine sourced hydrolyzed collagen; about 20%, about 25%, about 30%, about 35%, about 40%, about 45%, or about 50% by weight marine sourced hydrolyzed collagen; and about 0.5%, about 2%, about 4%, about 6%, about 8%, about 10%, about 12%, about 14%, about 16%, about 18%, about 20%, about 22%, about 24%, about 26% about 28%, about 30%, about 32%, about 34%, about 36%, about 38%, about 40%, or about 60% by weight whey.

The composition may be used to heal topical and/or internal wound sites. For example, the composition may be used prior to and after surgery to minimize cell damage and to expedite wound healing. The composition may be useful during surgery to foster separation of tissue to prevent adhesion formation. The composition may be used as a filler for a wound site and remain in the wound site as it heals, becoming part of the granulated tissue.

The composition may be useful for applications relating to cosmetic and plastic surgery, e.g., as a filler for lines and wrinkles formed in the skin.

The composition may take a physical form used in topical administration, such as a gel, spray, powder, paste, foam, film for incorporation in a dressing bandage, or a topically applied patch. The composition may take a physical form used in internal administration, such as an injectable liquid or an orally ingestible liquid.

The powder form will preferably have a moisture content of about 2-10 wt. % and a pH range of 5.5 to 6.5. The powder composition will have an ash content of less than 2.5 wt. % and an isotonic point of 5.0 to 6.5. In use, the powder composition may be the preferred physical form for use with irregularly shaped wounds. Tunnel wounds, flaps, and other non-conformative sites may be managed with the powder composition because it easily conforms to any shape wound, and may be applied by a poofer bottle or otherwise blown into difficult to reach wound sites. The powder is especially useful in wounds having a large amount of exudate, as the powder can absorb nearly 30 times its own weight. As the powder absorbs the exudate, a gel is formed, which completely fills the wound site, forming a mechanical barrier against bacterial infection. The powder does not exhibit the characteristic fly-away when being applied to the wound site, and administration is perfected due to the precise powder placement.

The gel form of the composition is especially useful in wounds with lesser amounts of exudate, in burns, and in surgical sites. Application of the gel can be dispensed through a tube, a syringe, or the reservoir in a topical patch. It is preferable to use about 60 wt. % collagen. The gel is formed by adding sterile water to collagen powder. The gel has the added advantage of adding moisture to the wound site, as well as inherent bacteriostatic properties, and stays positioned where applied.

A film form of the medicament composition may be made by mixing the powdered form with deionized water under heat at 155-175° F. Cross-linking and other agents, such as humectant, propylene glycol, sorbitol, and glycerine, may be added to the mixture. A preservative (such as benzyl alcohol or paraben) can be added. The mixture is cast on a belt liner by knife on a roll coating machine to form a liquid film, which is oven-dried. The film form can also be formed by cooling the liquid solution. These films can be used for drug or other chemical delivery, especially in dental applications. Antimicrobial and other medicinal agents can also be added to the film as needed for specific applications.

The composition may be formulated as a nutritional supplement. For example, at least one of vitamin A, vitamin C, vitamin E, vitamin B12, magnesium oxide, chelated manganese, grape seed extract, zinc, an alginate, cellulose, honey, chromium picolinate, selenium, glutamine, black seed oil, alpha lipoic acid, mulberry leaf extract, berberine, and glycosaminoglycans can be added to the composition to produce a nutrient composition for oral intake.

The composition can be sterilized by techniques known in the art, e.g., autoclaving, electron beam sterilization, ethylene oxide (EO) sterilization, and gamma sterilization. In addition to providing a sterile final product, sterilization can increase cross-linking, solubility, and/or humidity.

It is to be understood that the present composition is not limited to the embodiments described above, but encompasses any and all embodiments within the scope of the following claims.

I claim:

1. A composition for wound healing, comprising:
about 30% to about 90% by weight hydrolyzed collagen;
about 0.5% to about 60% by weight whey; and
magnesium stearate.

2. The composition for wound healing of claim 1, further comprising:
about 30% to about 90% by weight bovine sourced hydrolyzed collagen;
about 20% to about 50% by weight marine sourced hydrolyzed collagen;
about 0.5% to about 60% by weight whey; and
magnesium stearate.

3. The composition for wound healing of claim 2, wherein the whey is hydrolyzed whey.

4. The composition for wound healing of claim 2, wherein the marine sourced hydrolyzed collagen is sourced from salmon.

5. The composition for wound healing of claim 2, wherein the composition comprises:
about 60% to about 70% by weight bovine sourced hydrolyzed collagen;
about 20% by weight marine sourced hydrolyzed collagen;
about 10% to about 20% by weight whey; and
magnesium stearate;
wherein the marine sourced hydrolyzed collage in sourced from salmon.

6. The composition for wound healing of claim 1, further comprising a therapeutic agent.

7. The composition for wound healing of claim 6, wherein the therapeutic agent is selected from the group consisting of an antibiotic, glutamine, a glycosaminoglycan, zinc, an alginate, copper chlorophillan, fibronectin, silver, oxidized regenerated cellulose, cellulose, and honey.

8. The composition for wound healing of claim 7, wherein the glycosaminoglycan is selected from the group consisting of chondroitin sulfate, keratin sulfate, dermatic sulfate, hyaluronic acid, heparin, and heparin sulfate.

9. The composition for wound healing of claim 1, further comprising a cross-linking agent.

10. The composition for wound healing of claim 9, wherein the cross-linking agent is selected from the group consisting of a humectant, propylene glycol, sorbitol, and glycerine.

11. The composition for wound healing of claim 1, further comprising an antimicrobial.

12. The composition for wound healing of claim 11, wherein the antimicrobial is selected from the group consisting of tetracycline, streptomycin, cephalosporin, iodine, parachlorometaxylenol, and chlorhexidine.

13. The composition for wound healing of claim 1, further comprising a vitamin.

14. The composition for wound healing of claim 13, wherein the vitamin is selected from the group consisting of vitamin A, vitamin B12, vitamin C, and vitamin E.

15. The composition for wound healing of claim 1, further comprising an antioxidant.

16. The composition for wound healing of claim 15, wherein the antioxidant is selected from the group consisting of superoxide dismutase, glutathione peroxidase, and glutathione reductase.

17. The composition for wound healing of claim 1 further comprising an ingredient selected from the group consisting of black seed oil, alpha lipoic acid, mulberry leaf extract, berberine, lipoic acid, an omega compound, an omega-3 fatty acid compound, zeaxanthin, lutein, and hyaluronic acid.

18. A method of treating a wound, comprising:
administering to a patient a wound healing composition, the wound healing composition comprising a hydrolyzed collagen, a whey protein, and magnesium stearate.

19. The method of treating a wound of claim 18, wherein the wound healing composition comprises:
about 30% to about 90% by weight bovine sourced hydrolyzed collagen;
about 20% to about 50% by weight marine sourced hydrolyzed collagen;
about 0.5% to about 60% by weight whey; and
magnesium stearate.

20. The method of treating a wound of claim 18, wherein the wound healing composition comprises:
about 60% to about 70% by weight bovine sourced hydrolyzed collagen;
about 20% by weight marine sourced hydrolyzed collagen;
about 10% to about 20% by weight whey; and
magnesium stearate;
wherein the marine sourced hydrolyzed collage in sourced from salmon.
